(19) United States
(12) Patent Application Publication
(10) Pub. No.: US 2021/0030548 A1
(43) Pub. Date: Feb. 4, 2021

(54) INTERNAL OSSEOINTEGRATED IMPLANT FOR TRANSFEMORAL AMPUTEE

(71) Applicant: Thomas Cutler, Visalia, CA (US)

(72) Inventor: Thomas Cutler, Visalia, CA (US)

(21) Appl. No.: 16/984,064

(22) Filed: Aug. 3, 2020

Related U.S. Application Data
(60) Provisional application No. 62/882,430, filed on Aug. 2, 2019.

(51) Int. Cl. A61F 2/36; A61B 17/17

(52) U.S. Cl. CPC A61F 2/3609; A61B 17/17; A61F 2002/4205; A61F 2310/00005; A61F 2310/00023

(57) ABSTRACT

The present invention is an internal osseointegrated transfemoral amputee implant device and related methods. The device is designed to restore the enclosed nature of the bone marrow system which is disrupted by amputation, plus provide a pressure tolerant (weight-bearing) surface and mechanical anchoring for the iliotibial band.

### FIELD OF THE INVENTION

The present invention relates to implants for persons having an amputation, and more specifically to an internal implant for osseointegration into a trans-amputated bone of the amputee.

### DISCUSSION OF THE BACKGROUND

### Amputation

The American Academy of Orthopedic Surgeons (AAOS) promotes the Adductory Myodesis surgical technique. In this procedure, the surgical goals are primarily driven by a desire to prevent hip abduction contractures (the femur “sticking out to the side”; permanently angled away from midline of the body and unable to be positioned vertically or in anatomical position). The reaction to the purported problem of hip abduction contracture risk in transfemoral amputees is to tightly attach the thigh muscles on the inner thigh (hip adductors) and to transfer the attachment point of the iliotibial band from the lateral limb to the medial thigh.

This surgical technique is based on inaccurate assumptions regarding the function of the iliotibial band. The hip is a pivot upon which the body is balanced in gait. The hip abductors are the muscle group that supports the body weight in the hip joint. Hip abduction, therefore, is the act of supporting the body's mass. Hip abduction is thus desirable. Research has found that the iliotibial band is theoretically responsible for 30% of hip abduction torque. The oversight pertains to the fact that when hip abduction occurs from a muscle or tendon having a tibial insertion (pulling the tibia laterally below the knee), there is a reactive femoral adduction (pulling the femur medially above the knee). During transfemoral amputation, there is no longer a tibia to which the IT band may be attached since the limb is cut above the knee. This means that the reactive force which constrains the medial torque on the femur is removed, thus having the appearance of an unusually large hip adduction force. In actuality, the structure that had been previously masking the pre-existing dynamic femoral torque was removed, revealing the role of the IT band in this regard. Thus, conventional methods do not account for the fact that the IT band is a critical structure in hip abduction force.

Fat Embolism Syndrome is broadly seen as a concern in femoral fractures where the marrow from the bone is emitted into the surrounding tissue. It is associated with respiratory failure, neurocognitive deficit, and death. It is commonly overlooked in trauma situations. FES in transfemoral amputees can present an even greater long-term risk. Unlike fractures that eventually heal under normal circumstances, FES associated with amputation can be perpetual and with the compromised circulatory environment, the impact of FES can be magnified. Despite the awareness of the condition, there has been no research to date that has explored the extent of the risk nor the extent of the impact of FES on amputees. Thus, an open medullary canal is a significant risk to an amputee, but there has been no research to quantify the impact on amputees.

Early hip implants and knee implants were stainless-steel and cemented into place. Following the discovery of the titanium osseointegration, later implants were developed that integrated the benefits of direct osseointegration. Only percutaneous osseointegration has been applied to amputee prosthetic implants. It is believed that there are no internal applications of osseointegrated implants for amputees. In short, the discussion of osseointegration is limited to only those that go through the skin.

### SUMMARY OF THE INVENTION

The device of the present invention provides an internal osseointegrated prosthetic for femoral amputees that requires no traversing of the skin. Percutaneous osseointegration is unnecessary for most amputees. Only a minimally invasive anchoring system for the implant is necessary. Optimizing hip abduction torque via the iliotibial band is a benefit, not a detriment, to amputee function. Therefore, unlike other devices, the present amputee implant is not intended to be converted to a percutaneous version.

The device of the present invention is a surgically implanted osseointegrated prosthesis for transfemoral amputees that provides pressure tolerant skeletal weight bearing, provides a closure for the medullary canal system preventing bone marrow substances from leaking into the soft tissue in the area of the amputation, and provides a physical/mechanical anchor to which the iliotibial band may be optimally attached, and related methods. Unlike other amputee implants which are percutaneous, this implant is less invasive and only intended for internal applications.

The device of the present invention may include one or more of the following elements: a distal, weight bearing, elongated-dome-surface; a titanium stem for direct osseointegration of the device; an anterior iliotibial band attachment plate to anchor an IT band; an osseointegrated intramedullary implant limited in length to about 50 mm or less; a tapered stem for press-fitting; a posteriorly protruding spine for rotational control; and/or a weight bearing dome portion comprising a thermoplastic composition.

The device of the present invention provides a kind of “cap” for the femur. Rather than the implant extending deeply into the medullary canal of the femur, it can be effectively attached with an intramedullary portion that is about 2 inches (50 mm) or less in length. The device of the present invention is designed for use only with a transfemoral external socket that is connected to the limb. It provides a surgical foundation for improved prosthetic outcomes by providing a more appropriate residual limb. The patient and their rehabilitation team will assess the optimized limb and select from established prosthetic components, sockets, and suspension products. The implant may be osseointegrated and non-percutaneous.

The device of the present invention may have a rounded biocompatible (e.g., thermoplastic) dome-shaped weight bearing surface that is intentionally elongated rather than flattened in order to prevent lateral horizontal displacement of the femur within the soft tissues when the prosthesis is being used. Whereas a flattened distal surface would experience horizontal translation with hip abduction or adduction torque, a vertical elongation of the implant dome extending from the inferior surface would allow for a more intimate concave/convex fitting with less motion between the limb and the prosthetic socket.

Without limiting the invention, embodiments of the present invention may include a thermoplastic dome and a thermoplastic stem. The implant may include an anterior anchoring panel for anchoring the iliotibial band, the panel having fasteners (e.g., surgical screws, pins, etc.).

In some embodiments, the device may include a drilling guide for shaping a surface of the bone such that the surface of the bone is complementary to a shape of the stem. In some embodiments, the drilling guide comprises a plurality of approaches for shaping the surface of the bone. In some embodiments, the plurality of approaches comprises a first approach for forming a taper on the surface of the bone, and a second approach for forming a groove in the surface, the groove having a shape complementary to a shape of a protrusion of the stem. In some embodiments, the drilling guide comprises a central axis substantially parallel with a central axis of the bone. In such embodiments, the first approach will be a primary round taper and the second drill approach will provide the shape for a posterior protrusion of the stem. Embodiments of the present invention may include a tapered bone drilling attachment or attachments that will extrude bony material to form a shape in the distal femur that is complementary to that of the implant.

In some embodiments, the device may be transtibial or transfibular version. The transtibial and/or transfibular version may or may not include two appropriately smaller individual implants or may appear in the form of a bridged implant that fits into the medullary canal.

The present invention may also include tooling for shaping the host bone for insertion, as well as variations that apply to transtibial, transradial, and transhumeral amputation levels.

In some embodiments the present invention is directed to a method of implanting an implant device for a person having an amputation, the method comprising the steps of: providing the implant device, the implant device comprising a distal weight bearing head having an elongated, substantially convex shape, and a stem for insertion into a bone of the person, the stem comprising a porous material for osseointegration with the bone; inserting the stem into the bone; and closing the skin of the patient such that the entire implant device is under the skin. In some embodiments, the method further comprises the steps of: providing a drilling guide having at least one approach for shaping a surface of the bone; and shaping a surface of the bone using the drilling guide such that the surface comprises a shape complementary to a shape of the stem. In other embodiments, the method further comprises the steps of: providing a drilling guide having a first approach for drilling a tapered shape into a medullary cavity of the bone, the tapered shape being complementary to a shape of the stem, and a second approach for drilling a groove into the medullary cavity, the groove comprising a shape complementary to a protrusion of the stem for preventing twisting of the device relative to the bone, drilling the tapered shape into the medullary cavity, and drilling the groove into the medullary cavity.

### BRIEF DESCRIPTION OF THE DRAWINGS

FIG. 1 shows a view of an implant according to an embodiment of the present invention.

FIG. 2 shows a view of an implant according to an embodiment of the present invention.

### DETAILED DESCRIPTION OF THE EMBODIMENTS

Reference will now be made in detail to certain embodiments of the invention, examples of which are illustrated in the accompanying drawings. While the invention will be described in reference to these embodiments, it will be understood that they are not intended to limit the invention. To the contrary, the invention is intended to cover alternatives, modifications, and equivalents that are included within the spirit and scope of the invention as defined by the claims. In the following disclosure, specific details are given to provide a thorough understanding of the invention. However, it will be apparent to one skilled in the art that the present invention may be practiced without these specific details.

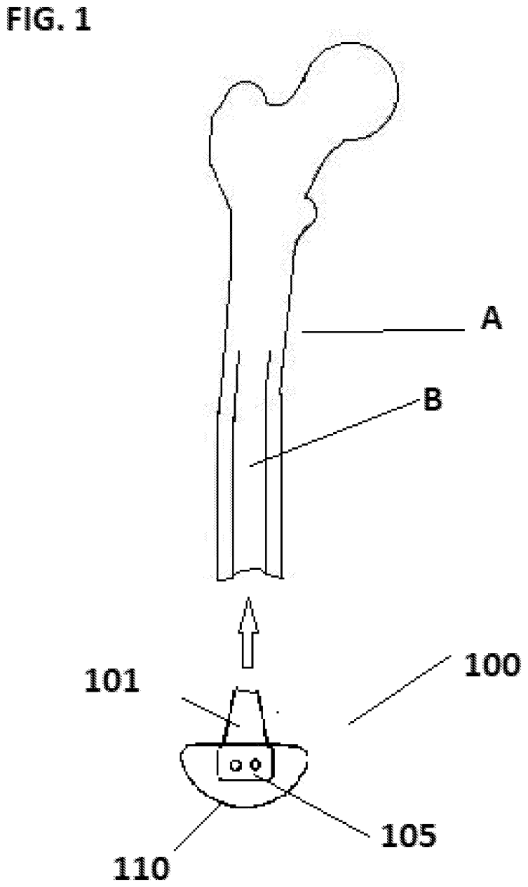

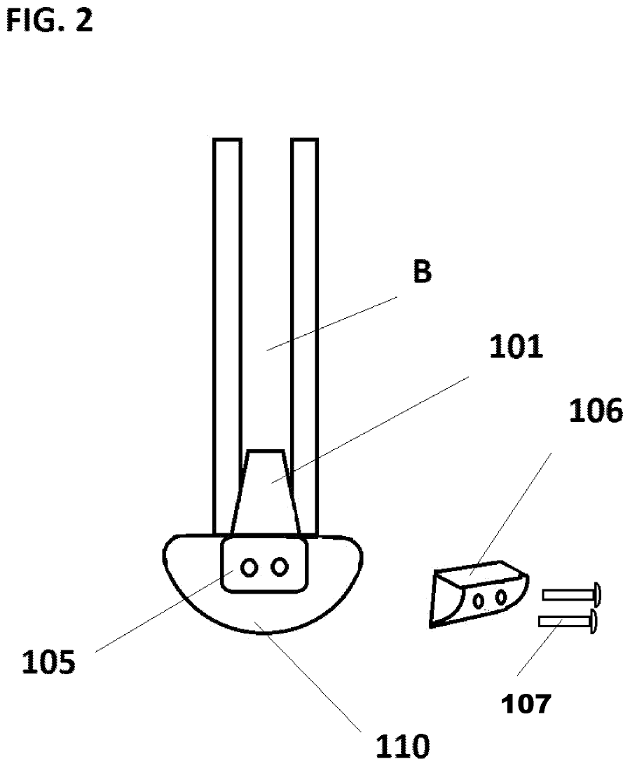

As shown in FIGS. 1-2, the present invention is directed to an implant device 100 for a person having an femoral amputation, the device 100 may include a distal, weight bearing head 110 having a substantially convex surface. In some embodiments, the weight bearing head 110 comprises a thermoplastic material. In some embodiments, the substantially convex surface is elongated to prevent lateral movement of the weight bearing head.

The weight bearing head may be attached to a stem 101 for insertion into a conditioned medullary cavity B of the amputee. In some embodiments, the stem 101 comprises a porous surface operable to become osseointegrated with the bone A. In some embodiments, the stem 101 comprises at least one of a tapered shape and a shape complementary to a natural shape of a medullary cavity B of the bone. In some embodiments, the stem 101 comprises a length of about 50 mm or less. In some embodiments, the stem 101 comprises a protrusion for preventing twisting of the stem in relation to the bone.

The device 100 may also include a soft tissue anchoring device 105 for attachment of an iliotibial band of the amputee. In some embodiments, the soft tissue attachment member 105 comprises a tab 106 having a shape complementary to a slot in the weight bearing head, the tab comprising at least one securing device 107 for securing the tab in the slot. In some embodiments, the securing device 107 comprises a screw or pin to secure the tab 106. In some embodiments, the soft tissue attachment member is operable to secure a tendon, a muscle, or another similar type of soft tissue of the person to the weight bearing head. The soft tissue anchoring mechanism may be for tethering the iliotibial band. This is done to provide additional hip abduction torque for the limb.

In some embodiments, the bone comprises a femur and the amputation comprises a transfemoral amputation. In some embodiments, the bone comprises at least one of a tibia, a fibula, a radius, an ulna, and a humerus, and the amputation comprises at least one of a transtibial, a transfibular, a transradial, a transulnar, and a transhumeral amputation. In some embodiments, the stem is inserted into a medullary cavity of the bone.

It should be understood that the foregoing descriptions of specific embodiments of the present invention have been presented for purposes of illustration and description. They are not intended to be exhaustive or to limit the invention to the precise forms disclosed, and obviously many modifications and variations are possible in light of the above teachings. The embodiments were chosen and described in order to best explain the principles of the invention and its practical application, and to thereby enable others skilled in the art to best utilize the invention and various embodiments with various modifications as are suited to the particular use contemplated.

What is claimed is:

1. An implant device for a person having an amputation, the device comprising:
a. a distal, weight bearing head having a substantially convex surface;
b. a stem for insertion into a bone of said amputee; and
c. a soft tissue attachment member,
wherein no portion of said device traverses the skin of said person.

2. The device of claim 1, wherein said substantially convex surface is elongated to prevent lateral movement of the weight bearing head.

3. The device of claim 1, wherein said weight bearing head comprises a thermoplastic material.

4. The device of claim 1, wherein said stem comprises a porous surface operable to become osseointegrated with said bone.

5. The device of claim 1, wherein said stem comprises a protrusion for preventing twisting of the stem in relation to the bone.

6. The device of claim 1, wherein said stem comprises at least one of titanium and a thermoplastic.

7. The device of claim 1, wherein said stem comprises at least one of a tapered shape and an shape complementary to a natural shape of a medullary cavity of said bone.

8. The device of claim 1, wherein said stem comprises a length of about 50 mm or less.

9. The device of claim 1, wherein said bone comprises a femur and said amputation comprises a transfemoral amputation.

10. The device of claim 1, wherein said bone comprises at least one of a tibia, a fibula, a radius, an ulna, and a humerus, and said amputation comprises at least one of a transtibial, a transfibular, a transradial, a transulnar, and a transhumeral amputation.

11. The device of claim 1, wherein said stem is inserted into a medullary cavity of said bone.

12. The device of claim 1, wherein said soft tissue attachment member comprises a tab having a shape complementary to a slot in said weight bearing head, said tab comprising at least one securing device for securing said tab in said slot.

13. The device of claim 12, wherein said securing device comprises a screw.

14. The device of claim 12, wherein said soft tissue attachment member is operable to secure a tendon, a muscle, or another similar type of soft tissue of said person to said weight bearing head.

15. The device of claim 1, further comprising a drilling guide for shaping a surface of said bone such that said surface of said bone is complementary to a shape of said stem.

16. The device of claim 15, wherein said drilling guide comprises a plurality of approaches for shaping said surface of said bone.

17. The device of claim 16, wherein said plurality of approaches comprises a first approach for forming a taper on said surface of said bone, and a second approach for forming a groove in said surface, said groove having a shape complementary to a shape of a protrusion of said stem.

18. The device of claim 15, wherein said drilling guide comprises a central axis substantially parallel with a central axis of said bone.

19. A method of implanting an implant device for a person having an amputation, the method comprising the steps of:
a. providing said implant device, said implant device comprising a distal weight bearing head having an elongated, substantially convex shape, and a stem for inserting into a bone of said person, said stem comprising a porous material for osseointegration with said bone;
b. inserting said stem into said bone; and
c. closing the skin of said patient such that the entire implant device is under said skin.

20. The method of claim 19, further comprising the steps of:
a. providing a drilling guide having at least one approach for shaping a surface of said bone; and
b. shaping a surface of said bone using said drilling guide such that said surface comprises a shape complementary to a shape of said stem.
